(19) United States
(12) Patent Application Publication
(10) Pub. No.: US 2012/0137752 A1
(43) Pub. Date: Jun. 7, 2012

(54) HYDROTESTING FLUIDS HAVING REDUCED MICROBIOLOGICALLY INFLUENCED CORROSION

(75) Inventor: Michael Morrow, Larkspur, CO (US)

(73) Assignees: CLEARWATER INTERNATIONAL, LLC; WEATHERFORD/LAMB, INC., Houston, TX (US)

(21) Appl. No.: 13/246,594

(22) Filed: Sep. 27, 2011

Related U.S. Application Data
(60) Provisional application No. 61/386,887, filed on Sep. 27, 2010.

(51) Int. Cl. G01M 3/02; C09K 15/04; C12N 1/20

(52) U.S. Cl. 73/49.5; 435/252.1; 435/252.2; 435/253.3; 435/252.8; 252/388; 252/408.1

(57) ABSTRACT

Hydrotesting fluid compositions including stable populations of corrosion reducing microbes, where the populations are is sufficient to reduce or eliminate microbiologically influenced corrosion (MIC).

### RELATED APPLICATIONS

This application claims the benefit of and priority to and is a non-provisional of U.S. Provisional Application Ser. No. 61/386,887 filed 27 Sep. 2010.

### BACKGROUND OF THE INVENTION

1. Field of the Invention

Embodiments of this invention relate to compositions and methods for treating seawater used as a hydrotesting fluid to eliminate the need to use toxic biocides.

More particularly, embodiments of this invention relate to compositions and methods for treating a hydrotesting fluid to eliminate the need to use toxic biocides, where the compositions include an effective population of a corrosion reducing microbe or a mixture of corrosion reducing microbes sufficient to reduce or eliminate microbiologically influenced or induced corrosion (MIC) of the hydrotesting fluid.

2. Description of the Related Art

A pipeline undergoing a hydrostatic test is vulnerable to oxygen corrosion and microbiologically influenced corrosion (MIC). To prevent oxygen corrosion, oxygen scavengers such as ammonium or sodium bisulfite are commonly used. Preventive measures for control of MIC normally involves treatment with chemical biocides. The use of biocides has proven to be effective in controlling MIC, however, most biocides add toxicity to the treated water. The toxicity of the water creates a predicament, because, at the completion of a hydrostatic test, the treated water must be discharged from the system, often with severe environmental constraints. For additional information about hydrotesting, the reader is referred to U.S. patent application Ser. Nos. 11/767,384, published as US20080314124 and 12/167,645, published as US20090067931, incorporated by reference through the operation of the closing clause of the specification.

This problem is being addressed by the industry by several means: (a) more stringent government regulation and legislation to control the type and concentration of chemicals that are allowed to be used offshore; (b) reducing the toxic effects of the effluent to the environment by further water treatment such as chemical neutralization, filtration, dilution, etc.; (c) alternative (non-biocide) chemical treatment, for example pH adjustment and sulfate ion removal; and (d) UV sterilization as an alternative treatment method to biocides.

While several methods are in use and under evaluation to reduce MIC, there is still a need in the art for more straightforward and environmentally acceptable methods and compositions for protecting pipelines during hydrotesting.

### SUMMARY OF THE INVENTION

Embodiments of the present invention provide hydrotesting fluid additive compositions including an effective amount of a nutrient system to encourage growth of corrosion reducing microbes in a hydrotesting fluid, where the effective amount produces a population of the corrosion reducing microbes in the hydrotesting fluid sufficient to reduce or eliminate microbiologically influenced or induced corrosion (MIC) of the hydrotesting fluid. Optionally, the additive compositions may include a seeding amount of corrosion reducing microbes.

Embodiments of the present invention provide hydrotesting fluid additive compositions including an effective amount of a nitrate-rich nutrient system to encourage growth of nitrate reducing bacteria (NRB) in a hydrotesting fluid, where the effective amount is sufficient to produce a population of one NRB or a mixture of NRBs in the hydrotesting fluid that reduces or eliminates microbiologically influenced corrosion (MIC) of the hydrotesting fluid. Optionally, the additive compositions may include a seeding amount of the NRB or the mixture of NRBs.

Embodiments of the present invention provide hydrotesting fluid additive compositions including an effective amount of a nutrient system to encourage growth of corrosion reducing microbes in a hydrotesting fluid, where the effective amount produces a population of one corrosion reducing microbe or a mixture of corrosion reducing microbes in the hydrotesting fluid sufficient to reduce or eliminate microbiologically influenced corrosion (MIC) of the hydrotesting fluid. Optionally, the additive compositions may include a seeding amount of the corrosion reducing microbe or the mixture of corrosion reducing microbes.

Embodiments of the present invention provide hydrotesting fluid compositions including an aqueous based fluid and an effective amount of a nitrate-rich nutrient system to encourage growth of a nitrate reducing bacteria (NRB) or a mixture of NRBs in the hydrotesting fluid, where the effective amount produces a population of the NRB or the mixture of NRBs in the hydrotesting fluid sufficient to reduce or eliminate microbiologically influenced corrosion (MIC) of the hydrotesting fluid. Optionally, the additive composition may include a seeding amount of the NRB or the mixture of NRBs.

Embodiments of the present invention provide a method for hydrotesting a pipeline or flowline including pumping into the pipeline or flow line a hydrotesting fluid composition including an aqueous based fluid and an effective amount of a nutrient system to encourage growth of a corrosion reducing microbe or a mixture of corrosion reducing microbes in the hydrotesting fluid, where the effective amount produces a population of the corrosion reducing microbe or the mixture of corrosion reducing microbes in the hydrotesting fluid sufficient to reduce or eliminate microbiologically influenced corrosion (MIC) of the hydrotesting fluid. Optionally, the additive composition may include a seeding amount of the corrosion reducing microbe or the mixture of corrosion reducing microbes. The adding may be intermittent, periodic, or based on an analysis of the hydrotesting fluid during testing.

Embodiments of the present invention provide a method for hydrotesting a pipeline or flowline including pumping into the pipeline or flow line a hydrotesting fluid composition including an aqueous based fluid and an effective amount of a nitrate-rich nutrient system to encourage growth of a nitrate reducing bacteria (NRB) or a mixture or NRBs in the hydrotesting fluid, where the effective amount produces a population of the NRB or the mixture ofNRBs in the hydrotesting fluid sufficient to reduce or eliminate microbiologically influenced corrosion (MIC) of the hydrotesting fluid. Optionally, the additive composition may include a seeding amount of the NRB or the mixture of NRBs. The adding may be intermittent, periodic, or based on an analysis of the hydrotesting fluid during testing.

### DETAILED DESCRIPTION OF THE INVENTION

The inventor has found that compositions for use in hydrotesting can be formulated to reduce or eliminate microbiologically influenced corrosion (MIC), thus reducing and/or eliminating the requirement to use toxic biocides. The inventor has found that by including an effective amount of a nutrient system to encourage growth of a corrosion reducing microbe or a mixture of corrosion reducing microbes in a hydrotesting fluid, where the effective amount produces a population of the corrosion reducing microbe or mixture of corrosion reducing microbes sufficient to reduce or eliminate microbiologically influenced or induced corrosion (MIC) of the hydrotesting fluid. In certain embodiments, the nutrient system is a nitrate-rich nutrient system adapted to promote the growth of a nitrate reducing bacteria (NRB) population in the hydrotesting fluid, where the NRB population is sufficient to reduce or eliminate microbiologically influenced corrosion (MIC). It is thought that a stable corrosion reducing microbe population such as a stable population of NRBs will reduce or eliminate populations of harmful souring bacteria such as sulfur-reducing bacterial (SRB) or other bacteria that produce corrosive agents such as hydrogen sulfide, thiols or other corrosive sulfur-containing agents. Although so-called nitrate biotechnology has been used to suppress the growth of SRB and to mitigate reservoir “souring” (due to generation of H₂S) in water flood operations, it has not been used to treat static, e.g. “non-flowing” hydrostatic test or hydrotesting fluids, especially hydrostatic or hyrdotesting of flow line or pipeline under static or non-flow conditions.

Hydrotesting of pipeline or flow lines using hydrotesting fluids including a microbial system of this invention is an alternative (non-biocide) treatment strategy for eliminating the use of chemical biocides in hydrotesting fluids. The microbial system and hydrotesting fluids derived therefrom are environmentally benign and safe to handle; there are no regulations on discharging the hydrotesting fluids of this invention offshore; and the hydrotesting fluids of this invention pose or show no adverse effect on marine life. Furthermore, it is estimated that hydrotesting fluids including the microbial system of this invention reduce or eliminate microbiologically influenced or induced corrosion (MIC) at a lower cost versus treatment with chemical biocides. The invention is based on microbial competitive growth. The hydrotesting fluids of this invention are designed to encourage the growth of corrosion reducing microbes at the expense of the growth of other bacteria known to produce corrosive agents such as hydrogen sulfide, thiols and other corrosive sulfur-containing species.

The present invention proposes to use nitrate biotechnology to treat seawater used as a hydrotest test fluid thus eliminating the requirement to use toxic biocides.

Embodiments of the present invention broadly relates to hydrotesting fluid additive compositions including an effective amount of a corrosion reducing microbe or a mixture of corrosion reducing microbes and optionally a nutrient system for the corrosion reducing microbe or the mixture of corrosion reducing microbes, where the effective amount is sufficient to reduce or eliminate microbiologically influenced corrosion (MIC). In certain embodiments, the additive reduces or eliminates the growth of sulfur-reducing bacteria (SRB) or other corrosion inducing microbes. In certain embodiments, the corrosion reducing microbe or the mixture of corrosion reducing microbes are a nitrate reducing bacteria (NRB) or a mixture of NRBs and the optional nutrient system is a nutrient system for the NRB or mixture of NRBs.

Embodiments of the present invention broadly relates hydrotesting fluid compositions including an aqueous based fluid, an effective amount of a corrosion reducing microbe or a mixture of corrosion reducing microbes and optionally a nutrient system for the corrosion reducing microbe or the mixture of corrosion reducing microbes, where the effective amount is sufficient to reduce or eliminate microbiologically influenced corrosion (MIC). In certain embodiments, the additive reduces or eliminates the growth of sulfur-reducing bacteria (SRB) or other corrosion inducing microbes. In certain embodiments, the corrosion reducing microbe or the mixture of corrosion reducing microbes are a nitrate reducing bacteria (NRB) or a mixture of NRBs and the optional nutrient system is a nutrient system for the NRB or mixture of NRBs. The fluids may also include a pH adjusting additive designed to maintain or adjust the pH of the fluid to a pH in the range of about pH 5 and about pH 8. In other embodiment, the pH is maintained in a range between about pH 5.5 and about pH 7.5. In other embodiment, the pH is maintained in a range between about pH 6.5 and about pH 7.5. In other embodiments, the pH is about 7.0. The term “about” here means that the pH is within about ±0.25 pH of the stated pH. In certain embodiments, the pH is within about ±0.2° C. of the stated pH. In other embodiments, the pH is within about ±0.1° C. of the stated pH.

Embodiments of the present invention broadly relates methods for hydrotesting a pipeline including pumping into the pipeline a hydrotesting fluid composition including an aqueous base fluid, an effective amount of a corrosion reducing microbe or a mixture of corrosion reducing microbes and optionally a nutrient system for the corrosion reducing microbe or the mixture of corrosion reducing microbes, where the effective amount is sufficient to reduce or eliminate microbiologically influenced corrosion (MIC). In certain embodiments, the additive reduces or eliminates the growth of sulfur-reducing bacteria (SRB) or other corrosion inducing microbes. In certain embodiments, the corrosion reducing microbe or the mixture of corrosion reducing microbes are a nitrate reducing bacteria (NRB) or a mixture of NRBs and the optional nutrient system is a nutrient system for the NRB or mixture of NRBs. The methods may also include adding microbe nutrient system during the hydrotesting to insure microbe growth. In certain embodiments, the microbes are NBRs and the nutrient system is an NBR nutrient system. The adding of the nutrient system maybe intermittent, periodic, or based on an analysis of the hydrotesting fluid during testing. In certain embodiments, the nutrient amount of the nutrient system is added at a rate sufficient to support a desired population of NBRs in the treating fluid. The methods may also include maintaining a pH of the fluid at a pH in the range of about pH 5 and about pH 8. In other embodiment, the pH is maintained in a range between about pH 5.5 and about pH 7.5. In other embodiment, the pH is maintained in a range between about pH 6.5 and about pH 7.5. In other embodiments, the pH is about 7.0. The term “about” here means that the pH is within about ±0.25 pH of the stated pH. In certain embodiments, the pH is within about ±0.2° C. of the stated pH. In other embodiments, the pH is within about ±0.1° C. of the stated pH. The methods may also include maintaining the temperature of the fluid at a temperature between about 10° C. and about 60° C. In certain embodiments, the fluid temperature is maintained between about 20° C. and about 50° C. In other embodiments, the fluid temperature is maintained between about 25° C. and about 45° C. In certain embodiments, the fluid temperature is maintained between about 25° C. and about 40° C. In certain embodiments, the fluid temperature is maintained between about 20° C. and about 35° C. In certain embodiments, the fluid temperature is maintained at about 30° C. The term “about” here means that the temperature is within about ±1° C. of the stated temperature. In certain embodiments, the temperature is within about ±0.5° C. of the stated temperature. In other embodiments, the temperature is within about ±0.1° C. of the stated temperature. In certain embodiments, the methods also include a means for stirring associated with the pigs or pipeline ends to produce a fluid circulation in the pipe line or flow line undergoing hydrotesting to improve nutrient and bacterial homogeneity or uniformity throughout the pipeline or flowline or portion thereof.

### Suitable Reagents

Suitable aqueous based fluids for use in the present invention include, without limitation, fresh water, salt water, phosphate brines, other brines, aqueous fluids including other additives, aqueous formate fluids, or mixtures and combinations thereof.

Suitable nitrate reducing bacteria for use in the present invention include, without limitation, Desulfovibrio sp. strain Lac3, Lac6 or Lac15 and Sulfurospirillum sp. strain KW, Shewanella, Desulfovibrio, e.g., Desulfovibrio desulfuricans; Geobacter metallireducens, Pseudomonas stutzeri and Paracoccus denitriricans, and species of Haloarcula, Halobacterium, Halofenax, Pyrobacterium, Aquasipirillum, Hyphomicrobium, Blastobacter, Hyphomicrobium, Cytophaga, Empedobacter, Azosipirillum, Flexibacter, Alteromonas, Magnetosipirillum, Pseudomonas, Achromobacter, Paracoccus, Alcaligenes, Bacillus, Alcaligenes, Agrobacterium, Campylobacter, Jonesia, Aquasipirillum, Eikenella, Azospirillum, Flavobacterium, Azoarcus, Kingella, Rhodobacter, Bacillus, Moraxella, Rhodopseudomonas, Bradyrhizobium, Morococcus, Rhodoplanes, Pseudomonas, Neisseria, Rhodobacter, Ochrobactrum, Rhodopseudomonas, Oligella, Beggiatoa, Sinorhizobium, Pseudomonas, Thiobacillus, Sphingobacterium, Thioploca, Aquifex, Tsukamurella, Bacillus, Ralstonia, Thermothrix, Paracoccus, Pseudomonas, Aquasipirillum, Halomonas, Nitrobacter, Nitrosomonas, Halomonas, Bacillus, Pseudomonas, Chromobacterium, Zavarzinia, Flavobacterium, or mixtures and combinations thereof. Exemplary examples of nitrogen reducing bacteria include, without limitation, (1) α Subclass of the Proteobacteria including Azospirillum brasilense strain DSM 1690, Agrobacterium tumefaciens DSM 30205, Rhodopseudomonas palustris DSM 123, Phototrophic Fe(II) oxidizer SW2, and Rhodobacter capsulatus DSM 152, (2) β Subclass of the Proteobacteria including Rubrivivax gelatinosus DSM 1709, Sphaerotilus natans DSM 565, Leptothrix discophora SS-1, Comamonas testosteroni DSM 50244, Alcaligenes eutrophus DSM 531, Fe(II) oxidizer BrG1, Fe(II) oxidizer BrG4, Fe(II) oxidizer BrG5, and Fe(II) oxidizer BrG2, (3) γ Subclass of the Proteobacteria including Pseudomonas putida DSM 50222, Pseudomonas stutzeri ATCC 14405, Xanthomonas fragariae DSM 3587, Escherichia coli DSM 498, Thiomicrospira pelophila DSM 1534, Thiomicrospira thyasirae DSM 5322, Thiomicrospira crunogena ATCC 35932, and Fe(II) oxidizer BrG3, (4) δ Subclass of the Proteobacteria including Desulfobulbus sp. DSM 2058, Desulfovibrio vulgaris DSM 644, Desulfovibrio baculatus DSM 2555, Desulfoarculus baarsii DSM 2075, Desulfobotulus sapovorans DSM 2055, Desulfobacter latus DSM 3381, Desulfobacter curvatus DSM 3379, (5) Cytophaga-Flavobacterium cluster including Cytophaga heparina DSM 2366, and Cytophaga johnsonae DSM 2064 and (6) mixtures thereof.

Suitable corrosion reducing microbe nutrients include, without limitation, a source of oxygen, a carbon source or other nutrients sufficient to support a desired population of corrosion reducing microbes.

Suitable NRB nutrients include, without limitation, nitrates and/or nitrites and optionally a carbon source such as methanol, a sugar, other similar nutrients or other nutrients needed to support a desired population of NRBs or mixtures thereof.

Suitable NRB cell populations for use in the hydrotesting fluid of this invention include, without limitation, the population has a cell density in the fluid between about 1×10² cells/mL to 1×10¹⁰ cells/mL. In certain embodiments, the population has a cell density in the fluid between about 1×10³ cells/mL to 1×10⁹ cells/mL. In certain embodiments, the population has a cell density in the fluid between about 1×10⁴ cells/mL to 1×10⁸ cells/mL. In certain embodiments, the population has a cell density in the fluid between about 1×10⁵ cells/mL to 1×10⁷ cells/mL.

Suitable nutrient system including, without limitation, Na₂SO₄, KH₂PO₄, NH₄Cl, MgCl₂.6H2O, KCl, CaCl₂.2H₂O, NaNO₃, 1 mL trace element solution SL10 (Widdel et al., 1983) and 0.5 mL 0.2% resazurin. An exemplary nutrient system for salt water or other sodium chloride brines includes 0.02 g Na₂SO₄, 1.0 g KH₂PO₄, 0.25 g NH₄Cl, 3.0 g MgCl₂.6H2O, 0.5 g KCl, 0.15 g CaCl₂.2H₂O, 0.7 gNaNO₃, 1 mL trace element solution SL10 (Widdel et al., 1983) and 0.5 mL 0.2% resazurin. The nutrient system may also include a carbon source such as methanol, ethanol, a sugar, a mixture of sugars, other carbon sources or mixtures thereof.

### Embodiments of the Invention

A hydrotesting fluid additive compositions including an effective amount of a microbe selected from the group consisting of (1) α Subclass of the Proteobacteria including Azospirillum brasilense strain DSM 1690, Agrobacterium tumefaciens DSM 30205, Rhodopseudomonas palustris DSM 123, Phototrophic Fe(II) oxidizer SW2, and Rhodobacter capsulatus DSM 152, (2) β Subclass of the Proteobacteria including Rubrivivax gelatinosus DSM 1709, Sphaerotilus natans DSM 565, Leptothrix discophora SS-1, Comamonas testosteroni DSM 50244, Alcaligenes eutrophus DSM 531, Fe(II) oxidizer BrG1, Fe(II) oxidizer BrG4, Fe(II) oxidizer BrG5, and Fe(II) oxidizer BrG2, (3) γ Subclass of the Proteobacteria including Pseudomonas putida DSM 50222, Pseudomonas stutzeri ATCC 14405, Xanthomonas fragariae DSM 3587, Escherichia coli DSM 498, Thiomicrospira pelophila DSM 1534, Thiomicrospira thyasirae DSM 5322, Thiomicrospira crunogena ATCC 35932, and Fe(II) oxidizer BrG3, (4) δ Subclass of the Proteobacteria including Desulfobulbus sp. DSM 2058, Desulfovibrio vulgaris DSM 644, Desulfovibrio baculatus DSM 2555, Desulfoarculus baarsii DSM 2075, Desulfobotulus sapovorans DSM 2055, Desulfobacter latus DSM 3381, Desulfobacter curvatus DSM 3379, (5) Cytophaga-Flavobacterium cluster including Cytophaga heparina DSM 2366, and Cytophaga johnsonae DSM 2064 and (6) mixtures thereof and optionally a nutrient system for the corrosion reducing microbe or the mixture of corrosion reducing microbes, where the effective amount is sufficient to reduce or eliminate microbiologically influenced corrosion (MIC). In certain embodiments, the additive reduces or eliminates the growth of sulfur-reducing bacteria (SRB) or other corrosion inducing microbes. In certain embodiments, the corrosion reducing microbe or the mixture of corrosion reducing microbes are a nitrate reducing bacteria (NRB) or a mixture of NRBs and the optional nutrient system is a nutrient system for the NRB or mixture of NRBs.

Embodiments of the present invention broadly relates hydrotesting fluid compositions including an aqueous based fluid, an effective amount of a microbe selected from the group consisting of (1) α Subclass of the Proteobacteria including Azospirillum brasilense strain DSM 1690, Agrobacterium tumefaciens DSM 30205, Rhodopseudomonas palustris DSM 123, Phototrophic Fe(II) oxidizer SW2, and Rhodobacter capsulatus DSM 152, (2) β Subclass of the Proteobacteria including Rubrivivax gelatinosus DSM 1709, Sphaerotilus natans DSM 565, Leptothrix discophora SS-1, Comamonas testosteroni DSM 50244, Alcaligenes eutrophus DSM 531, Fe(II) oxidizer BrG1, Fe(II) oxidizer BrG4, Fe(II) oxidizer BrG5, and Fe(II) oxidizer BrG2, (3) γ Subclass of the Proteobacteria including Pseudomonas putida DSM 50222, Pseudomonas stutzeri ATCC 14405, Xanthomonas fragariae DSM 3587, Escherichia coli DSM 498, Thiomicrospira pelophila DSM 1534, Thiomicrospira thyasirae DSM 5322, Thiomicrospira crunogena ATCC 35932, and Fe(II) oxidizer BrG3, (4) δ Subclass of the Proteobacteria including Desulfobulbus sp. DSM 2058, Desulfovibrio vulgaris DSM 644, Desulfovibrio baculatus DSM 2555, Desulfoarculus baarsii DSM 2075, Desulfobotulus sapovorans DSM 2055, Desulfobacter latus DSM 3381, Desulfobacter curvatus DSM 3379, (5) Cytophaga-Flavobacterium cluster including Cytophaga heparina DSM 2366, and Cytophaga johnsonae DSM 2064 and (6) mixtures thereof and optionally a nutrient system for the corrosion reducing microbe or the mixture of corrosion reducing microbes, where the effective amount is sufficient to reduce or eliminate microbiologically influenced corrosion (MIC). In certain embodiments, the additive reduces or eliminates the growth of sulfur-reducing bacteria (SRB) or other corrosion inducing microbes. In certain embodiments, the corrosion reducing microbe or the mixture of corrosion reducing microbes are a nitrate reducing bacteria (NRB) or a mixture of NRBs and the optional nutrient system is a nutrient system for the NRB or mixture of NRBs. The fluids may also include a pH adjusting additive designed to maintain or adjust the pH of the fluid to a pH in the range of about pH 5 and about pH 8. In other embodiment, the pH is maintained in a range between about pH 5.5 and about pH 7.5. In other embodiment, the pH is maintained in a range between about pH 6.5 and about pH 7.5. In other embodiments, the pH is about 7.0. The term “about” here means that the pH is within about ±0.25 pH of the stated pH. In certain embodiments, the pH is within about ±0.2° C. of the stated pH. In other embodiments, the pH is within about ±0.1° C. of the stated pH.

Embodiments of the present invention broadly relates methods for hydrotesting a pipeline including pumping into the pipeline a hydrotesting fluid composition including an aqueous base fluid, an effective amount of a corrosion reducing microbe microbes selected from the group consisting of (1) α Subclass of the Proteobacteria including Azospirillum brasilense strain DSM 1690, Agrobacterium tumefaciens DSM 30205, Rhodopseudomonas palustris DSM 123, Phototrophic Fe(II) oxidizer SW2, and Rhodobacter capsulatus DSM 152, (2) β Subclass of the Proteobacteria including Rubrivivax gelatinosus DSM 1709, Sphaerotilus natans DSM 565, Leptothrix discophora SS-1, Comamonas testosteroni DSM 50244, Alcaligenes eutrophus DSM 531, Fe(II) oxidizer BrG1, Fe(II) oxidizer BrG4, Fe(II) oxidizer BrG5, and Fe(II) oxidizer BrG2, (3) γ Subclass of the Proteobacteria including Pseudomonas putida DSM 50222, Pseudomonas stutzeri ATCC 14405, Xanthomonas fragariae DSM 3587, Escherichia coli DSM 498, Thiomicrospira pelophila DSM 1534, Thiomicrospira thyasirae DSM 5322, Thiomicrospira crunogena ATCC 35932, and Fe(II) oxidizer BrG3, (4) δ Subclass of the Proteobacteria including Desulfobulbus sp. DSM 2058, Desulfovibrio vulgaris DSM 644, Desulfovibrio baculatus DSM 2555, Desulfoarculus baarsii DSM 2075, Desulfobotulus sapovorans DSM 2055, Desulfobacter latus DSM 3381, Desulfobacter curvatus DSM 3379, (5) Cytophaga-Flavobacterium cluster including Cytophaga heparina DSM 2366, and Cytophaga johnsonae DSM 2064 and (6) mixtures thereof and optionally a nutrient system for the corrosion reducing microbe or the mixture of corrosion reducing microbes, where the effective amount is sufficient to reduce or eliminate microbiologically influenced corrosion (MIC). In certain embodiments, the additive reduces or eliminates the growth of sulfur-reducing bacteria (SRB) or other corrosion inducing microbes. In certain embodiments, the corrosion reducing microbe or the mixture of corrosion reducing microbes are a nitrate reducing bacteria (NRB) or a mixture of NRBs and the optional nutrient system is a nutrient system for the NRB or mixture of NRBs. The methods may also include adding microbe nutrient system during the hydrotesting to insure microbe growth. In certain embodiments, the microbes are NBRs and the nutrient system is an NBR nutrient system. The adding of the nutrient system may be intermittent, periodic, or based on an analysis of the hydrotesting fluid during testing. In certain embodiments, the nutrient amount of the nutrient system is added at a rate sufficient to support a desired population of NBRs in the treating fluid. The methods may also include maintaining a pH of the fluid at a pH in the range of about pH 5 and about pH 8. In other embodiment, the pH is maintained in a range between about pH 5.5 and about pH 7.5. In other embodiment, the pH is maintained in a range between about pH 6.5 and about pH 7.5. In other embodiments, the pH is about 7.0. The term “about” here means that the pH is within about ±0.25 pH of the stated pH. In certain embodiments, the pH is within about ±0.2° C. of the stated pH. In other embodiments, the pH is within about ±0.1° C. of the stated pH. The methods may also include maintaining the temperature of the fluid at a temperature between about 10° C. and about 60° C. In certain embodiments, the fluid temperature is maintained between about 20° C. and about 50° C. In other embodiments, the fluid temperature is maintained between about 25° C. and about 45° C. In certain embodiments, the fluid temperature is maintained between about 25° C. and about 40° C. In certain embodiments, the fluid temperature is maintained between about 20° C. and about 35° C. In certain embodiments, the fluid temperature is maintained at about 30° C. The term “about” here means that the temperature is within about ±1° C. of the stated temperature. In certain embodiments, the temperature is within about ±0.5° C. of the stated temperature. In other embodiments, the temperature is within about ±0.1° C. of the stated temperature. In certain embodiments, the methods also include a means for stirring associated with the pigs or pipeline ends to produce a fluid circulation in the pipe line or flow line undergoing hydrotesting to improve nutrient and bacterial homogeneity or uniformity throughout the pipeline or flowline or portion thereof. The temperature maybe maintained either using a heating associated with the pigs used to isolate the pipeline or flowline portion or associated with the ends of the pipeline or flowline.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

We claim:

1. A hydrotesting additive composition comprising:
an effective amount of a corrosion reducing microbe or a mixture of corrosion reducing microbes, and
optionally a growth effective amount of a nutrient system for the corrosion reducing microbe or the mixture of corrosion reducing microbes,
where the effective amount is sufficient to reduce or eliminate microbiologically influenced corrosion (MIC) in an aqueous hydrotreating fluid.

2. The composition of claim 1, wherein the additive composition retards, reduces or stops the growth of sulfur-reducing bacteria (SRB).

3. The composition of claim 1, wherein the corrosion reducing microbe or a mixture of corrosion reducing microbes comprise a nitrate reducing bacteria (NRB) or a mixture of NRBs and the nutrient system is for the NRB or the mixture NRBs.

4. The composition of claim 1, wherein the aqueous hydrotreating fluid is selected from the group consisting of fresh water, salt water, phosphate brines, other brines, aqueous fluids including other additives, aqueous formate fluids, and mixtures thereof.

5. The composition of claim 1, wherein the microbes are selected from the group consisting of (1) α Subclass of the Proteobacteria including Azospirillum brasilense strain DSM 1690, Agrobacterium tumefaciens DSM 30205, Rhodopseudomonas palustris DSM 123, Phototrophic Fe(II) oxidizer SW2, and Rhodobacter capsulatus DSM 152, (2) β Subclass of the Proteobacteria including Rubrivivax gelatinosus DSM 1709, Sphaerotilus natans DSM 565, Leptothrix discophora SS-1, Comamonas testosteroni DSM 50244, Alcaligenes eutrophus DSM 531, Fe(II) oxidizer BrG1, Fe(II) oxidizer BrG4, Fe(II) oxidizer BrG5, and Fe(II) oxidizer BrG2, (3) γ Subclass of the Proteobacteria including Pseudomonas putida DSM 50222, Pseudomonas stutzeri ATCC 14405, Xanthomonas fragariae DSM 3587, Escherichia coli DSM 498, Thiomicrospira pelophila DSM 1534, Thiomicrospira thyasirae DSM 5322, Thiomicrospira crunogena ATCC 35932, and Fe(II) oxidizer BrG3, (4) δ Subclass of the Proteobacteria including Desulfobulbus sp. DSM 2058, Desulfovibrio vulgaris DSM 644, Desulfovibrio baculatus DSM 2555, Desulfoarculus baarsii DSM 2075, Desulfobotulus sapovorans DSM 2055, Desulfobacter latus DSM 3381, Desulfobacter curvatus DSM 3379, (5) Cytophaga-Flavobacterium cluster including Cytophaga heparina DSM 2366, and Cytophaga johnsonae DSM 2064 and (6) mixtures thereof.

6. A hydrotesting composition comprising:
an aqueous base fluid,
an effective amount of a corrosion reducing microbe or a mixture of corrosion reducing microbes,
optionally a nutrient system for the corrosion reducing microbe or a mixture of corrosion reducing microbes, and
optionally a pH adjusting system for adjusting the pH of the fluid to a pH between about pH 5 and pH 8,
where the effective amount is sufficient to reduce or eliminate microbiologically influenced corrosion (MIC).

7. The composition of claim 6, wherein the additive composition retards, reduces or stops the growth of sulfur-reducing bacteria (SRB).

8. The composition of claim 6, wherein the corrosion reducing microbe or a mixture of corrosion reducing microbes comprise a nitrate reducing bacteria (NRB) or a mixture of NRBs and the nutrient system is for the NRB or the mixture NRBs.

9. The composition of claim 6, wherein the aqueous hydrotreating fluid is selected from the group consisting of fresh water, salt water, phosphate brines, other brines, aqueous fluids including other additives, aqueous formate fluids, and mixtures thereof.

10. The composition of claim 1, wherein the microbes are selected from the group consisting of (1) α Subclass of the Proteobacteria including Azospirillum brasilense strain DSM 1690, Agrobacterium tumefaciens DSM 30205, Rhodopseudomonas palustris DSM 123, Phototrophic Fe(II) oxidizer SW2, and Rhodobacter capsulatus DSM 152, (2) β Subclass of the Proteobacteria including Rubrivivax gelatinosus DSM 1709, Sphaerotilus natans DSM 565, Leptothrix discophora SS-1, Comamonas testosteroni DSM 50244, Alcaligenes eutrophus DSM 531, Fe(II) oxidizer BrG1, Fe(II) oxidizer BrG4, Fe(II) oxidizer BrG5, and Fe(II) oxidizer BrG2, (3) γ Subclass of the Proteobacteria including Pseudomonas putida DSM 50222, Pseudomonas stutzeri ATCC 14405, Xanthomonas fragariae DSM 3587, Escherichia coli DSM 498, Thiomicrospira pelophila DSM 1534, Thiomicrospira thyasirae DSM 5322, Thiomicrospira crunogena ATCC 35932, and Fe(II) oxidizer BrG3, (4) δ Subclass of the Proteobacteria including Desulfobulbus sp. DSM 2058, Desulfovibrio vulgaris DSM 644, Desulfovibrio baculatus DSM 2555, Desulfoarculus baarsii DSM 2075, Desulfobotulus sapovorans DSM 2055, Desulfobacter latus DSM 3381, Desulfobacter curvatus DSM 3379, (5) Cytophaga-Flavobacterium cluster including Cytophaga heparina DSM 2366, and Cytophaga johnsonae DSM 2064 and (6) mixtures thereof.

11. A method for hydrotesting a pipeline comprising:
pumping into a pipeline, a flow line or a portion thereof a hydrotesting composition including:
an aqueous base fluid,
an effective amount of a corrosion reducing microbe or a mixture of corrosion reducing microbes, and
optionally a nutrient system for the corrosion reducing microbe or a mixture of corrosion reducing microbes,
where the effective amount is sufficient to reduce or eliminate microbiologically influenced corrosion (MIC) and
testing the pressure of the pipeline, flowline or portion thereof for a reduction in pressure indicating a leak.

12. The method of claim 10, further comprising:
adding an additional amount of the corrosion reducing microbe or the mixture of corrosion reducing microbes on an intermittent basis, periodic basis, or based on an analysis of the hydrotesting fluid during testing to maintain a desired population of cells in the fluid and/or a desired amount of the nutrient system in the fluid.

13. The method of claim 10, further comprising:
adding a pH adjusting amount of a pH adjusting system of an intermittent basis, periodic basis, or based on an analysis of the hydrotesting fluid during testing to maintain a pH between about pH 5 and pH 8.

14. The method of claim 10, further comprising:
maintaining the fluid at a temperature between about 10° C. and about 60° C.

15. The method of claim 10, wherein the additive composition retards, reduces or stops the growth of sulfur-reducing bacteria (SRB).

16. The method of claim 10, wherein the corrosion reducing microbe or a mixture of corrosion reducing microbes comprise a nitrate reducing bacteria (NRB) or a mixture ofNRBs and the nutrient system is for the NRB or the mixture NRBs.

17. The method of claim 10, wherein the aqueous hydrotreating fluid is selected from the group consisting of fresh water, salt water, phosphate brines, other brines, aqueous fluids including other additives, aqueous formate fluids, and mixtures thereof.

18. The method of claim 10, wherein the microbes are selected from the group consisting of (1) α Subclass of the Proteobacteria including Azospirillum brasilense strain DSM 1690, Agrobacterium tumefaciens DSM 30205, Rhodopseudomonas palustris DSM 123, Phototrophic Fe(II) oxidizer SW2, and Rhodobacter capsulatus DSM 152, (2) β Subclass of the Proteobacteria including Rubrivivax gelatinosus DSM 1709, Sphaerotilus natans DSM 565, Leptothrix discophora SS-1, Comamonas testosteroni DSM 50244, Alcaligenes eutrophus DSM 531, Fe(II) oxidizer BrG1, Fe(II) oxidizer BrG4, Fe(II) oxidizer BrG5, and Fe(II) oxidizer BrG2, (3) γ Subclass of the Proteobacteria including Pseudomonas putida DSM 50222, Pseudomonas stutzeri ATCC 14405, Xanthomonas fragariae DSM 3587, Escherichia coli DSM 498, Thiomicrospira pelophila DSM 1534, Thiomicrospira thyasirae DSM 5322, Thiomicrospira crunogena ATCC 35932, and Fe(II) oxidizer BrG3, (4) δ Subclass of the Proteobacteria including Desulfobulbus sp. DSM 2058, Desulfovibrio vulgaris DSM 644, Desulfovibrio baculatus DSM 2555, Desulfoarculus baarsii DSM 2075, Desulfobotulus sapovorans DSM 2055, Desulfobacter latus DSM 3381, Desulfobacter curvatus DSM 3379, (5) Cytophaga-Flavobacterium cluster including Cytophaga heparina DSM 2366, and Cytophaga johnsonae DSM 2064 and (6) mixtures thereof.
